(19) United States
(12) Patent Application Publication
(10) Pub. No.: US 2014/0274796 A1
(43) Pub. Date: Sep. 18, 2014

(54) Methods, Systems, and Compositions for In Vitro Concentric Cell Culture Analog Systems

(71) Applicant: University of Central Florida Research Foundation, Inc., Orlando, FL (US)

(72) Inventor: James J. Hickman, Orlando, FL (US)

(73) Assignee: University of Central Florida Research Foundation, Inc., Orlando, FL (US)

(21) Appl. No.: 14/214,822

(22) Filed: Mar. 15, 2014

Related U.S. Application Data
(60) Provisional application No. 61/789,587, filed on Mar. 15, 2013.

(51) Int. Cl. G01N 33/50

(52) U.S. Cl. CPC G01N 33/5014
USPC 506/10; 506/39; 506/14

(57) ABSTRACT

The present invention comprises methods, systems and compositions comprising concentric chamber cell culture analog devices, comprising biologically functional cells, which function similarly to in vivo conditions.

### CROSS-REFERENCE TO RELATED APPLICATION

The present application claims the benefit of the U.S. Provisional Application No. 61/789,587 filed Mar. 15, 2013.

### STATEMENT REGARDING FEDERALLY SPONSORED RESEARCH OR DEVELOPMENT

This invention was made with government support under Grant No. R01 EB005459 and Grant No. UH2TR000516 awarded by National Institutes of Health. The government has certain rights in the invention.

### BACKGROUND OF THE INVENTION

Disclosed herein are methods, systems, and compositions relating to in vitro cell culture devices to mimic mammalian organ systems.

The major research uses of animals are both in assessing potential toxicity of chemicals and in drug testing. Animal tests often are long in duration, expensive, and raise ethical issues. Further, animal tests are not always predictive of human response. This fact is easily demonstrated in drug development where only 11% of chemicals exiting animal trials are successful in humans [Hughes 2007]. In terms of human response to environmental toxicants, it is not ethically possible to conduct direct tests on humans, and extrapolation of animal results to human response is problematic. Over-regulation results in unnecessary expense and under-regulation endangers human health and the environment, so better testing systems are necessary.

In vitro tests can supplement and may reduce dependency on animal tests. However, current in vitro tests fail to capture many important aspects of human and mammalian response to chemicals. Most in vitro tests are based on the use of multi-well plates where isolated cells or tissues are placed in medium spiked with a bolus dose of the test chemical. Such systems miss key aspects of physiological response. For example, the dose dynamics in the body differ considerably from traditional systems as time-dependent changes in chemical concentration occur in the body at a tissue site due to the processes controlling absorption, distribution, metabolism and excretion of a compound. Further, traditional well systems typically use a single cell or tissue type; in the body, metabolites are exchanged between different tissue/organ compartments. This is a serious detriment of traditional well systems as interorgan interactions in vivo are a well-known consideration. As blood flows in a sequence from one organ to another, the products which result from the activity of one particular organ can affect other organs, causing either toxicity or detoxification, from case to case. An example of such an interorgan effect is a liver-derived metabolite called naphthalene, which causes lung toxicity. Similarly, cardiac toxicity in rats can occur upon exposure to a metabolite of cyclophosphamide, known as acrolein, which is also formed in the liver.

What is needed in the art is a cell culture analog device and methods and systems thereof, comprising biologically functioning cells that mimic interactions of organs and whole organism systems.

### BRIEF SUMMARY OF THE INVENTION

Disclosed are methods, systems, and compositions comprising concentric chamber cell culture analog devices, comprising biologically functional cells, which function similarly to in vivo conditions. For example, a component region of a device can comprise cardiac myocytes on microelectrode arrays.

Disclosed are concentric chamber cell culture analog systems or arrays comprising one or more chambers or components. A component can comprise a concentric chamber cell culture device, which is also referred to as a concentric chamber cell culture analog device comprising cells. A component, with or without cells contained within a chamber, and/or other elements, is analogous to an organ or organ system. A component can comprise a container for cells, such as a chamber, in which cells are contained, grown, acted on, and/or maintained in the chamber. For example, a component can comprise, but is not limited to, a cardiac component comprising patterned biologically functional cardiac myocytes on microelectrode arrays, a hepatic component comprising liver cells, a gastrointestinal component comprising cells such as epithelial cells and/or mucus-producing cells, a muscular component comprising muscle cells, a kidney-like filtering component, an “other tissues” component, a neural component, or other components analogous to body structures, organs, or organ systems.

Further disclosed are methods for determining the effect of an input variable on one or more components or cultures of cells, comprising contacting the one or more components or cultures of cells with at least one input variable and monitoring at least one output parameter. For example, one or more components or cultures of cells can be used for example, in a non-limiting listing, the testing of compounds, infectious agents, immune responses, cellular factors, hormones, molecules, gases, and environmental effects on in vitro whole body systems (such as pressure or atmospheric changes), or other conditions.

Disclosed herein is a concentric chamber cell culture analog system or an array of components, comprising a plurality of concentric chambers (components), for example, wherein one or more components is a component comprising patterned biologically functional cardiac myocytes on microelectrode arrays, a hepatic component comprising liver cells, a gastrointestinal component comprising epithelial cells and/or mucus-producing cells, a muscular component comprising muscle cells, a kidney-like filtering component, an “other tissues” component, a neural component, and/or other component analogous to body structures, organs, or organ systems, and optionally, further comprising housing for enclosing the component. An “other tissues” compartment represents and is analogous to fluid retained in nonabsorbing, nonadsorbing, and/or nonmetabolizing tissues that captures the dynamics of exposure to a chemical in the concentric chamber cell culture analog systems.

Disclosed herein are methods, systems, and means for dynamically observing a concentric chamber cell culture analog system, for example, comprising a computer and other elements, such as processors, sensors, actuators, etc., wherein, in an aspect, a disclosed method comprises analyzing data from a plurality of sensors to measure physiological events in one or more chambers of one or more components disclosed herein; optionally, regulating a cell culture characteristic such as temperature, light, oxygen, carbon dioxide, and/or fluid mixing rates of a culture medium in at least one chamber of a component; and detecting biological or toxicological reactions in the cells or other elements of one or more chambers of a component; and optionally, upon detection, recording the change and/or changing one or more parameters of a component.

Disclosed herein is a computer-readable medium having computer-executable instructions stored thereon to perform a method. For example, a disclosed method can comprise analyzing data from a plurality of sensors to measure physiological events in one or more chambers of one or more components disclosed herein; optionally, regulating a characteristic such as temperature, light, oxygen, carbon dioxide, and/or fluid mixing rates of a culture medium in at least one chamber of a component; and detecting biological or toxicological reactions in the cells or other elements of one or more chambers of a component; and optionally, upon detection, recording the change and/or changing one or more parameters of one or more components.

### BRIEF DESCRIPTION OF THEN SEVERAL VIEWS OF THE DRAWINGS

The accompanying Figures, which are incorporated in and constitute a part of this specification, illustrate several aspects and together with the description serve to explain the principles of the invention.

FIG. 1A-FIG. 1B show two cell type seeding chambers formed within 2 concentric PDMS rings affixed on a commercially available micro-electrode array chip.

FIG. 2A-FIG. 2C show HepG2 cells seeded in the outer chamber (A), rat cardiomyocytes patterns cultured on the top of electrodes in the inner chamber (B), and signals from one micro-electrode array recorded after 10 days in culture (C).

FIG. 3A-FIG. 3C shows HepG2 human cell line hepatocytes seeded in the outer chamber (A), human cardiomyocytes cells cultured in the inner chamber (B), and signal from one micro-electrode array recorded after 8 days in culture (C) (in which spontaneous rhythmic activity occurred after 1 day in culture).

### DETAILED DESCRIPTION OF THE INVENTION

Disclosed herein is an in vitro model of mammalian response to chemicals or chemical mixtures. Such a model reduces dependency on animal testing while providing improved predictions of responses of human or other organisms, such as plants, animals, or insects. Disclosed herein are concentric chamber cell culture analog methods, systems, and devices. Disclosed methods, systems, and devices can comprise microfabrication techniques and cell culture/tissue engineering. A concentric chamber cell culture analog device, also referred to herein as “a concentric chamber cell culture analog system device” or a component, is a physical representation of a physiologically based interorgan interaction model, and the functional in vitro systems reproduce in vivo effects including, but not limited to, toxicity and detoxification responses, cardiac pacemaking, muscle dynamics, and neuronal information processing.

Disclosed herein are cell culture analog systems comprising cells grown in a concentric chamber cell culture analog device comprising one or more components, chambers, or regions, wherein a component, along with cells contained therein the chamber, and/or other elements, is analogous to an organ or organ system. A disclosed component comprises a container for cells, or a chamber, in which cells are contained, grown, acted on, and/or maintained in the component. For example, a component can comprise, but is not limited to, a cardiac component comprising patterned biologically functional cardiac myocytes on microelectrode arrays. See U.S. patent application Ser. No. 12/938,701, which is incorporated by reference herein in its entirety for disclosing patterned rat cardiomyocyte cultures on microelectrode arrays in a serum-free medium for the study of cardiac physiology and pharmacology, utilizing a high-throughput technique. A disclosed component can comprise a support substrate, wherein the surface of the support substrate can be modified to determine and/or enhance cell attachment. Surface modification can be done either by traditional protein absorption method or using self-assembled monolayers (SAMs) and may use extracellular matrix components such as fibronectin, collagen or organo silanes containing amine moieties such as DETA (trimethoxysilylpropyldiethylenetriamine).

In an aspect, a disclosed component can comprise a support substrate bearing a multielectrode array (MEA) and a negative surface resistant to cell attachment and can be deposited on the support substrate covering the MEA. The negative surface can bear a pattern ablated on it by, for example, laser photolithography. A positive surface promoting cell attachment can be deposited on the pattern ablated on the negative surface and cardiomyocytes adherent to the positive surface and growing aligned along the pattern. This application (U.S. application Ser. No. 12/938,701) also teaches methods of making the culture of patterned cardiomyocytes. For example, a method can comprise preparing a support substrate bearing a MEA, and overlaying on the support substrate a negative surface resistant to cell adherence. The surface can comprise polyethylene glycol covering the MEA. Further, a disclosed method can comprise ablating a pattern on the negative surface, depositing on the ablated pattern a positive surface promoting cell adherence and including fibronectin, adhering cardiomyocytes on the positive surface, and culturing the cardiomyocytes to grow on the positive patterned surface and aligned with it.

In an aspect, a component or chamber can comprise a muscular component comprising muscle cells. See U.S. patent application Ser. No. 12/765,399, which is incorporated by reference herein in its entirety for disclosing methods for lengthening the useful life of a culture of muscles cells by using disclosed mixtures of serum-free media, supplemented with growth factors. Tables 1 and 2 show the individual growth factors, hormones, and neurotransmitters that support muscle and neuromuscular junction development. For example, the composition shown in Table 1 is a formulation for a serum-free medium for culturing motor neurons with adult spinal cord neurons. Table 2 lists additional factors identified in muscle development and neuromuscular junction formation. NBactiv4, used for maintenance of the cells, improves the survival of the skeletal muscle cells.

In an aspect, a disclosed component or chamber can comprise a neural component. See U.S. patent application Ser. No. 12/117,339, which is incorporated by reference herein in its entirety for disclosing a method of culturing adult mammalian spinal cord neurons so that they exhibit electrical functionality. Table 3 shows a non-limiting example of a serum-free culture medium used in a disclosed method.

In an aspect, a disclosed component or chamber can comprise a kidney-like filtering region, an “other tissues” region, and/or other regions analogous to body structures, organs, or organ systems.

In an aspect, a disclosed system, component, or chamber can comprise a hepatic component comprising liver cells, or a gastrointestinal component comprising epithelial cells and/or mucus-producing cells.

Disclosed herein are methods for determining the effect of an input variable on a culture system of cells, comprising contacting the concentric chamber cell culture system with an input variable and monitoring at least one output parameter.

Disclosed herein is an array of disclosed components or disclosed chambers, comprising a plurality of components, for example, comprising one or more of patterned biologically functional cardiac myocytes on microelectrode arrays, a hepatic component comprising liver cells, a gastrointestinal component comprising epithelial cells and/or mucus-producing cells, a muscular component comprising muscle cells, a kidney-like filtering component, an other tissues region, a neural component, and/or other components analogous to body structures, organs, or organ systems, and optionally, further comprising housing for enclosing the components.

Disclosed herein is an in vitro chamber of a component or array, analogous to heart function in organisms, such as a human, animal, or insect, comprising cardiac myocytes, surface embedded microelectrodes, and patterned substrates on the microelectrode array to monitor the condition of the cardiac chamber in the concentric chamber cell culture analog device in real time and detect both acute and chronic functional toxic effects on the system.

Cultured cardiac myocytes are widely used in toxin detection and in drug development to screen for unwanted cardiac side effects [Meyer 2004]. Cardiac myocytes are whole-cell biosensors as they are spontaneously active, can be kept in culture in stable conditions for extended periods [Dhir 2009], and respond to a wide spectrum of known and unknown toxins. Patterning cardiac myocytes on microelectrode arrays allows for the measurement of more advanced parameters, such as reverse use dependence, variability in QT interval, and relative refractory periods [Natarajan 2011].

A disclosed concentric chamber cell culture analog system can comprise a plurality of chambers or components, which provide in vitro reproduction or simulation of a living body or interorgan interaction, with each chamber or component representing an organ or tissue. Disclosed systems and devices can be used for research, testing, diagnosis and insight into underlying biochemical mechanisms and how function is affected. By inserting functional tissues into chambers comprising mammalian cells or tissues, response from exposure to active agents, such as environmental chemicals, can be measured.

Disclosed components, chambers, arrays and methods can be used with any type of culturable cells including, but not limited to, both animal cells and human cells, or other known cells types to insects, and methods can comprise cross-species extrapolation. The disclosed components, arrays, and methods can be used in studies on naphthalene toxicity on drug combinations to treat multidrug resistant cancer [Tatosain 2009] or colon cancer and on hormone disruptors. Additionally, disclosed components, arrays, and methods can comprise cardiac, neuronal, muscle, and neuromuscular junction systems. See U.S. patent application Ser. No. 12/765,996, which is incorporated by reference herein in its entirety for disclosing long term in vitro cultures of tissue engineered functional neuromuscular junctions. Tables 1, 2, and 3 provide examples of the composition of serum-free medium that can be used in disclosed methods. See also U.S. patent application Ser. No. 13/102,672, which is incorporated by reference herein in its entirety for disclosing the formation of neuromuscular junctions in a defined system by co-culturing one or more human motor neuron cells and one or more rat muscle cells in a substantially serum-free medium. Tables 1, 2, and 3 represent non-limiting examples of serum-free media used in disclosed methods.

For the validation of the integrated cardiac myocyte reporter construct and a concentric chamber cell culture analog device, the effect of metabolism on the functional effects of stereoisomers of permethrin, which is a pyrethroid and an environmental toxin, can be measured. The role of enantioselectivity in environmental safety is poorly understood for pesticides, and the knowledge gap is reflected in that the great majority of chiral pesticides are used and regulated as if they were achiral, that is, single compounds [Liu 2005]. Stereoisomerism is critically important for pyrethroid toxicity; it determines not only their efficacy on their main target, but more importantly, their metabolic rate. The disclosed components, arrays, and methods are ideal in vitro systems to study the effect of metabolism on the effect of environmental toxins in a system that is adaptable to a high-throughput format. For example, an in vitro system that allows for the observation of functional units derived from human cells/tissues is advantageous for environmental toxin studies. In a non-limiting example, human stem cells can be used for more authentic constructs leading to human-based components, arrays, and methods. Thus, described herein are components, arrays, and methods using specific organ systems represented by in vitro models, including, but not limited to, for example, a cardiac analog using patterned cardiac myocytes. The analogs described contemplate organ or tissue analogs found in human, animal, plant, or insect bodies. Particular examples are not to be seen as limitations of the present invention.

Incorporation of a functional cardiac system in a concentric chamber cell culture analog device to form a component enables the discovery of complex, unknown, and unexpected effects of active agents, such as toxicants, pharmaceuticals, infectious agents, cellular factors, antibodies, and other stimuli or environmental factors. Reverse use dependence, variability in QT intervals, and relative refractory period (which is related to triangulation) are measured in an in vitro system based on patterned cardiac myocytes. The in vitro electrophysiological measurement parameters are analogous to the parameters used in the SCREENIT scoring system introduced by Hondeghem and coworkers in 1994 [Carlsson 2006]. In that model, variability in action potential (AP) duration, triangulation of the repolarization phase of the AP and reverse use dependence is measured on female rabbit Langendorff-perfused hearts. This in vitro system does not reproduce the whole complexity of the heart, but shows that the measured parameters are able to measure the most important arrhythmogenic mechanisms including rhythm generation (chronotropy, firing frequency dispersion), conduction (conduction velocity, conduction velocity dispersion, frequency dependence of conduction velocity), and re-entry (QT interval, QT interval dispersion, reverse use dependence, absolute and relative refractory period). These parameters have high predictive value for cardiac side effects. In addition, by utilizing a serum-free, defined culture medium, as disclosed herein—for example, in Tables 1-3, one of the major unknown variables in the system would be removed.

The disclosed systems and methods can comprise cells. Cells include but are not limited to, animal, human, plant, or insect cells. Disclosed cells can provide data that can reduce dependency on animals for testing and provide insights that cannot be obtained from whole animals. The present invention can lead to a more accurate and cost-effective assessment of the toxicological potential of environment chemicals or chemical mixtures. In an aspect, “cell culture analogs” (CCA) can be combined with the development of functional tissue mimics. These approaches are combined to make a realistic in vitro model of a mammal and predict its response from exposure to a chemical or chemical mixture, referred to herein as an active agent, whether particularly active on a cell or not. Disclosed systems, arrays, and components can comprise functional muscle as well as neuronal systems. Human stem cells can be used for more authentic constructs leading to a human based components, arrays and methods.

Disclosed herein is a physical representation of a physiological based interorgan interaction. Interorgan interaction in vivo is well known, since blood flows in a sequence from one organ to another. Metabolic products that result from the activity of one particular organ can affect other organs, potentially resulting in toxicity or detoxification, depending on the active agent. An example of such an interorgan effect is a liver-derived metabolite called naphthalene, which causes lung toxicity. Similarly, cardiac toxicity in rats can occur upon exposure to a metabolite of cyclophosphamide, known as acrolein, which is formed from cyclophosphamide in the liver.

While pharmacokinetic computational models have proven to be useful aids in studies of absorption, distribution, metabolism, elimination, and toxicity (ADMET), they are limited. All relevant reactions and physiological responses must be identified, particularly molecular mechanisms underlying cell response. For complex systems, such as mammals, it is difficult to capture not only the primary reactions but also all of the secondary responses (e.g., the metabolite of A, made in the liver, circulates to another tissue causing the release of B which then causes other cells to change physiologically). The disclosed components, arrays and methods compensate for this lack of complete knowledge.

In addition to the limitations of current in vitro tests to predict systemic effects, most assays are based on single cell-type analysis. It is well known that single cell-types are limited in their ability to mimic in vivo tissue function. Functional cellular models, or multi-cellular systems that allow evaluation of properties previously only possible in intact animals or organs such as muscle dynamics [Wilson 2010], cardiac pacemaking [Natarajan 2011], neuronal function [Varghese 2010] and neuromuscular junction (NMJ) function [Guo 2010], have been developed to overcome these limitations but have not as yet been integrated. The disclosed components, arrays, and methods can provide a combination of these functional in vitro systems into a system that more accurately recapitulates the human response.

A disclosed device can comprise at least two concentric chambers bonded to a substrate. The area of the first chamber is defined by its walls, which can be any geometric shape having a circumference or perimeter, a diameter, and a height. The second concentric chamber can have a circumference, diameter, and height that are larger than the first chamber, such that the first chamber is a discrete well within a larger well. The area of the second chamber can comprises the space between the walls of the first chamber and the walls of the second chamber. Additional chambers can be added sequentially, such that a plurality of chambers of increasing circumferences or perimeter can be constructed. The number of chambers can be altered depending on the application.

In an aspect, the walls of the chambers can have increasing heights and diameters such that the outer chambers have larger diameters and taller walls than those concentric chambers within. The height of the walls can increase with each sequential concentric chamber outside of the first chamber, such that when the chambers are filled with a fluid medium, the concentric chambers can communicate with one another through the fluid connection above the walls. The diameter, height, and number of the chambers can be adjusted depending on the application.

In an aspect, the walls of the chambers can have variable heights. For example, the first chamber and second chamber can have walls of equal heights, while a third chamber has walls that are taller than the first and second chamber. The variable heights of sequential concentric chambers can be adjusted depending on the application.

The surface substrate of the communicating concentric chambers can be modified to determine and enhance cell attachment. At least a portion of the surface substrate modification can be done either by traditional protein absorption method or using self-assembled monolayers (SAMs) and can use extracellular matrix components such as fibronectin, collagen or organo silanes containing amine moieties such as DETA (trimethoxysilylpropyldiethylenetriamine). Additionally, the surface substrate can comprise a multielectrode array (MEA).

In an aspect, portions of the surface substrate can be surface modified in various ways, such that different chambers can be modified in different ways. For example, the first chamber can comprise MEAs and the second chamber can comprise SAMs. In a further aspect, fractions within a chamber can be surface modified in different ways. For example, half of the first chamber can comprise MEAs and the other half of the first chamber can comprise SAMs.

In a non-limiting example, a disclosed device can comprise two concentric polydimethylsiloxane (PDMS) rings oxygen plasma bonded on a hard substrate as shown in FIG. 1. The PDMS rings can have different heights and diameters—the outside one being taller than the inside one, such that the two of them can communicate at the top. The inside ring forms one chamber, and the space between the outer and inner rings forms the second chamber. Both the height and diameter of the rings can be adjusted depending on the application.

Cells can be cultured on the surface of the surface-modified substrate of the device using standard cell culture equipment and methods. In this “well within a well” system, the cells can be physically separated by walls, for example PDMS walls, but since the medium is shared at through the liquid connection above the chamber walls, the device can be used to assess inter-cell type interactions. In addition, the chamber walls, for example PDMS rings, can be plasma bonded on the top of a substrate containing embedded extracellular electrodes, which allows field potential recordings from electrically active cells, such as cardiomyocytes or neurons. Further, the device can be extended to a multi-well within a well system, where the central ring can be the shortest and the smallest in diameter, while the sequential concentric chambers can be increasing both in height and diameter to the outermost one. Rings of various sizes can be cast in custom made molds. Mixing of the medium contained in the system can be done by placing the device on a rocking platform or another comparable device.

An advantage of disclosed components, arrays, and methods is that they are inexpensive to make and can support high throughput studies. Unlike other in vitro systems, such as multi-well plates, the disclosed components, arrays, and methods provide realistic dose dynamics (similar to what occurs in an animal or human) and allow for the formation and exchange of metabolites between compartments or chambers as well as exchange of compounds induced by the presence of the parental compound or metabolites. The present invention can be used to test underlying molecular mechanisms of interorgan interactions.

Disclosed components, arrays, and methods are more advantageous than traditional static culture systems as concentric chamber cell culture analog devices are amenable to high-throughput analysis and the addition of biologically functional tissues in the present invention, such as patterned cardiac myocytes integrated with microelectrode arrays (MEAs), increases the information control and allows for the use of electrical measurements to monitor response, while maintaining the ability to observe more traditional endpoints in concert.

Disclosed herein are monitoring methods, which include, but are not limited to, non-invasive, more high throughput, high information content, functional, able to detect known and unknown effects of active agents at physiological concentrations, appropriate for continuous monitoring, compatible with fluidic systems, and mechanically robust. Hybrid (live-cell/electronic) systems have been developed to overcome several shortcomings of traditional whole-cell biosensors, at the same time preserving their advantageous properties over traditional physico-chemical or biochemical sensing methods [Natarajan 2006; Jung 1998; Wilson 2007; Wilson 2010, Varghese 2010; Molnar 2007; Wilson 2011].

Disclosed herein is a method comprising cardiac myocytes. In an aspect, cardiac myocytes can be cultured on surface embedded microelectrodes and patterned substrates on the microelectrode array to monitor the condition of the cardiac chamber in a device of the present invention in real time. Cultured cardiac myocytes are widely used in toxin detection and in drug development to screen for unwanted cardiac side effects [Meyer 2004]. It has been shown that pyrethroids [Natarajan 2006] and heavy metals can be detected, and in some extent classified, based on their physiological effects on the spontaneous activity of cultured cardiac myocytes measured using a non-invasive, high-throughput, chronic protocol with substrate-embedded MEAs.

Disclosed herein are components, arrays, and methods comprising one or more liver cells. In an aspect, liver cells can comprise a liver analog region to mimic metabolism. Liver cells that can be used in the disclosed methods, systems, and components can include, but are not limited to, HepG2 cells, differentiated embryonic stem cells, differentiated adult pluripotent stem cells, Fa2N-4 cells, and HepaRG cells.

In an aspect, disclosed herein are components, arrays, and methods comprising a liver cells, a patterned cardiac myocyte/MEA functional reporter region, and “other tissues” regions. For example, the disclosed components, arrays, and methods can be used to validate the integrated cardiac myocyte reporter region and the functional effects of stereoisomers of permethrin (a pyrethroid which is an environmental toxin) on the tissues in the system can be measured. Permethrin has four stereospecific isomers: 1R-cis-, 1R-trans-, 1S-cis-, and 1S-trans-. The 1R-cis- and 1R-trans-isomers are active, whereas the other two are not [Lund 1982]. Moreover, the cis isomers are about ten times more toxic than the trans isomers in vivo. Recent data indicated that the metabolic rate of cis-permethrin is much slower than that of the trans isoform, which could be an explanation for the different in vivo toxicity [Scollon 2009].

Disclosed herein are components, arrays, and methods that can be used to determine and measure the effect of different enantiomers, for example permethrin, on spontaneous beating and conduction velocity of patterned cardiac myocytes in the presence and absence of one or more chambers of a component representing the major metabolic pathways in the body. The lifetime of components can be extended to examine the effects of a compound in chronic studies.

Disclosed herein are components, arrays, and methods comprising patterned biologically functional cardiac myocytes on microelectrode arrays and other chambers comprising cells, structures, factors, co-factors or other elements for constructing analogs of organ tissues or systems that mimic physiological, physical, chemical, and/or electrical conditions of whole organ systems or organisms.

Disclosed herein are methods for determining the effect of an input variable, comprising contacting cells comprised by one or more components or chambers with an input variable and monitoring at least one output parameter. For example, components, arrays and methods can comprise testing of active agents for beneficial or deleterious effects, long-term studies of exposure to active agents, determination of active metabolites or other studies designed by those skilled in the art using the components, arrays and methods of the present invention.

Disclosed herein are devices, arrays, and/or systems comprising a housing for enclosing a device, arrays and/or systems disclosed herein, at least a device comprising a plurality of chambers, or a “well within a well” system, wherein cells can be physically separated by chamber walls, where the first chamber can be the shortest and the smallest in diameter, while the sequential concentric chambers can be increasing both in height and diameter to the outermost one, such that the medium is shared at the top of the chamber walls. The device can be used to assess inter-cell and/or interorgan interactions. In addition, the chamber walls can be plasma bonded on the top of a substrate containing embedded extracellular electrodes, which allows field potential recordings from electrically active cells, such as cardiomyocytes or neurons.

In an aspect, the heights of the chamber walls can be variable. For example, the first chamber can have walls measuring 0.5 cm, the second and third chambers can have walls measuring 0.75 cm, the fourth chamber can have walls measuring 1.0 cm, and the fifth chamber can have walls measuring 1.25 cm. The height of the chamber walls can be adjusted depending on the application and the type of cells being cultured in a specific chamber.

Disclosed herein are methods, systems, and means for dynamically observing a concentric chamber cell culture analog system, for example, comprising a computer and other elements, such as processors, sensors, actuators, etc., wherein, in an aspect, a method comprises analyzing data from a plurality of sensors to measure physiological events in one or more chambers of one or more components disclosed herein; optionally, regulating a cell culture characteristic such as temperature, light, oxygen, carbon dioxide, and/or fluid mixing rates of a culture medium in at least one chamber of a component; and detecting biological or toxicological reactions in the cells or other elements of one or more chambers of a component; and optionally, upon detection, recording the change and/or changing one or more parameters of a component.

Disclosed herein is a computer-readable medium having computer-executable instructions stored thereon to perform a method. For example, a disclosed method can comprise analyzing data from a plurality of sensors to measure physiological events in one or more chambers or components disclosed herein; optionally, regulating a characteristic such as temperature, light, oxygen, carbon dioxide, and/or fluid mixing rates of a culture medium in at least one chamber of a component; and detecting biological or toxicological reactions in the cells or other elements of one or more chambers of a component; and optionally, upon detection, recording the change and/or changing one or more parameters of component.

Disclosed herein is a component device, comprising one or more chambers and optionally, cells; and one or more sensing elements, wherein a chamber, and optionally with the cells contained within or on the chamber, and/or other elements, simulates interorgan interactions, physiological, physical, chemical, and/or electrical conditions of whole organs, tissues or organisms.

Disclosed herein is a component device, comprising one or more chambers and one or more sensing elements, wherein a chamber and/or sensing elements simulates one or more interorgan interactions, a physiological condition, a physical condition, a chemical condition, and/or a electrical condition of one or more whole organs, tissues, or organisms. In an aspect, a disclosed chamber comprises cells.

In an aspect, a disclosed component device can comprise sensors, alarms, or computer control elements, or a combination thereof. In an aspect of a disclosed component device, interorgan interactions, physiological, physical, chemical, or electrical conditions, or a combination thereof can be monitored in real time.

In an aspect, cells can be derived from a human, or an animal, or a plant, or an insect. In an aspect, cells can be a combination of cells, such as, for example, a combination of cells from a human, or an animal, or a plant, or an insect. In an aspect, cells can be a mixture of cells, such as, for example, a mixture of cells from a human, or an animal, or a plant, or an insect. In an aspect, cells can be derived from a human.

In an aspect, a disclosed device component can comprise a chip comprising biological cells on a microelectrode array comprising surface embedded microelectrodes.

In an aspect, a disclosed device component can comprise at least a first chamber containing a first type of cell under conditions where the first type of cell provides at least one parameter value comparable to a value obtained for the same type of cell in vivo, wherein the first chamber is concentrically contained within a second chamber containing a second type of cell under conditions where the second type of cell provides at least one parameter value comparable to a value obtained for the same type of cell in vivo. In an aspect of a disclosed component device, a second chamber can be concentrically contained within a third chamber containing a second type of cell under conditions where the third type of cell provides at least one parameter value comparable to a value obtained for the same type of cell in vivo. In an aspect of a disclosed device, a third chamber can be concentrically contained within a fourth chamber containing a fourth type of cell under conditions where the third type of cell provides at least one parameter value comparable to a value obtained for the same type of cell in vivo. In an aspect of a disclosed device, a fourth chamber can be concentrically contained within a fifth chamber containing a fifth type of cell under conditions where the third type of cell provides at least one parameter value comparable to a value obtained for the same type of cell in vivo.

In an aspect, at least one device can comprise a first chamber comprising a first cell type maintained under conditions providing at least one parameter value comparable to values obtained for the cells in vivo; a second chamber of the same or different geometry than the first chamber comprising a second cell type maintained under conditions providing at least one parameter value comparable to values obtained for the cells in vivo; wherein the first and second chambers are interconnected by a fluidic connection about the chamber wall of the first chamber.

In an aspect, a disclosed component device can comprise one or more additional chambers containing the same or different types of cells as in the first or second chambers, under conditions where the additional cell provides at least one parameter value comparable to a value obtained for the same type of cell in vivo, wherein the one or more additional chambers are interconnected by a fluidic connection about the chamber walls of the interior chambers.

In an aspect, a disclosed component device can comprise culture medium. In an aspect, culture medium can be serum-free. In an aspect of a disclosed component device, at least one interorgan interaction can be measured by determining one or more reactions by cells to an active agent, one or more reactions by cells to an input parameter, interaction between cells, liquid residence time, liquid to cell ratio, metabolism by cells, or shear stress. In an aspect, a disclosed component device can provide for three-dimensional growth of cells. In an aspect, at least one of the chambers can comprise a tissue biopsy. In an aspect, at least one of the chambers can comprise a cross-section of a tissue.

In an aspect, a disclosed device can be used for high throughput studies. In an aspect, a disclosed device can be used in chronic toxicity studies. In an aspect, a disclosed device can be operated for 72 hours, 84 hours, 96 hours, 108 hours, 120 hours, 132 hours, 144 hours, 156 hours, 168 hours, 180 hours, or for days or weeks, or longer, or any amount of time in between.

Disclosed herein is an array of components, comprising one or more culture analog system devices comprising one or more chambers, and optionally, cells; and one or more sensing elements, wherein a chamber, and optionally with the cells contained within or on the chamber and/or other elements, simulates interorgan interaction, physiological, physical, chemical, and/or electrical conditions of whole organs, tissues or organisms.

Disclosed herein is an array of components, comprising one or more culture analog system devices comprising one or more chambers and one or more sensing elements, wherein a chamber and/or a sensing element simulates at least one interorgan interaction, physiological condition, physical condition, chemical condition, and/or electrical condition of one or more whole organs, tissues, or organisms. In an aspect, a disclosed chamber can comprise cells.

In an aspect, a disclosed array of components can comprise connection elements, filters, sensors, alarms, or computer control elements, or a combination thereof. In an aspect, at least one interorgan interaction, physiological, physical, chemical, and/or electrical condition can be monitored in real time. In an aspect, cells can be derived from a human, or an animal, or a plant, or an insect. In an aspect, cells can be a combination of cells, such as, for example, a combination of cells from a human, or an animal, or a plant, or an insect. In an aspect, cells can be a mixture of cells, such as, for example, a mixture of cells from a human, or an animal, or a plant, or an insect. In an aspect, cells can be derived from a human.

In an aspect of a disclosed array of components, at least one device can comprise a chip comprising biological cells on a microelectrode array comprising surface embedded microelectrodes. In an aspect, a disclosed device can comprise at least a first chamber containing a first type of cell under conditions where the first type of cell provides at least one parameter value comparable to a value obtained for the same type of cell in vivo, wherein the first chamber is concentrically contained within a second chamber containing a second type of cell under conditions where the second type of cell provides at least one parameter value comparable to a value obtained for the same type of cell in vivo. In an aspect, a second chamber can be concentrically contained within a third chamber containing a second type of cell under conditions where the third type of cell provides at least one parameter value comparable to a value obtained for the same type of cell in vivo. In an aspect, a third chamber can be concentrically contained within a fourth chamber containing a fourth type of cell under conditions where the third type of cell provides at least one parameter value comparable to a value obtained for the same type of cell in vivo. In an aspect, a fourth chamber can be concentrically contained within a fifth chamber containing a fifth type of cell under conditions where the third type of cell provides at least one parameter value comparable to a value obtained for the same type of cell in vivo.

In an aspect of a disclosed array of components, at least one device can comprise a first chamber comprising a first cell type maintained under conditions providing at least one parameter value comparable to values obtained for the cells in vivo; a second chamber of the same or different geometry than the first chamber comprising a second cell type maintained under conditions providing at least one parameter value comparable to values obtained for the cells in vivo; wherein the first and second chambers are interconnected by a fluidic connection about the chamber wall of the first chamber. In an aspect, a disclosed array of components can comprise one or more additional chambers containing the same or different types of cells as in the first or second chambers, under conditions where the additional cell provides at least one parameter value comparable to a value obtained for the same type of cell in vivo, wherein the one or more additional chambers are interconnected by a fluidic connection about the chamber walls of the interior chambers.

In an aspect, a disclosed array of components can comprise at least one culture medium. In an aspect, at least one culture medium can be serum-free. In an aspect, at least one of the interorgan interactions can be measured by determining one or more reactions by cells to an active agent, one or more reactions by cells to an input parameter, interaction between cells, liquid residence time, liquid to cell ratio, metabolism by cells, or shear stress.

In an aspect, a disclosed array of components can be used for high throughput studies. In an aspect, at least one disclosed device of the array can be used for high throughput studies. In an aspect, a disclosed array can be used in chronic toxicity studies. In an aspect, at least one disclosed device of the array can be used in chronic toxicity studies. In an aspect, a disclosed device can be operated for 72 hours, 84 hours, 96 hours, 108 hours, 120 hours, 132 hours, 144 hours, 156 hours, 168 hours, 180 hours, or for days or weeks, or longer, or any amount of time in between.

Disclosed herein is a method for determining the effect of an input variable on a culture system of cells, comprising contacting at least one cell of a component or array with an input variable and monitoring at least one output parameter. In an aspect, a step of monitoring at least one output parameter can comprise obtaining information from a sensor in a chamber, chip, or region, or from one or more devices in an array, or from a combination thereof. In an aspect of a disclosed method, an input variable can be an organic compound. In an aspect, an input variable can be an inorganic compound. In an aspect, an input variable can be a complex sample. In an aspect, an input variable can be a pharmaceutical, environmental sample, a nutritional sample, or a consumer product. In an aspect, an input variable can be a virus, liposome, nanoparticle, biodegradable polymer, radiolabeled particle or toxin, biomolecule, toxin-conjugated particle, or biomolecule.

In an aspect, a disclosed method can comprise high throughput studies. In an aspect, a disclosed method can comprise chronic toxicity studies. In an aspect, a disclosed method can be executed for 72 hours, 84 hours, 96 hours, 108 hours, 120 hours, 132 hours, 144 hours, 156 hours, 168 hours, 180 hours, or for days or weeks, or longer, or any amount of time in between.

Disclosed herein is a computerized method for dynamically controlling a component, comprising, detecting data from one or more sensors in a component device, and analyzing data from one or more sensors to measure physiological events in one or more chambers of a component. In an aspect, a report of the data and/or analysis can be provided. In an aspect, a disclosed computerized method can comprise regulating one or more parameters of the device or detecting biological or toxicological reactions in the chambers of the component device; and optionally, upon detection, changing one or more pharmacokinetic parameters of a disclosed component device. In an aspect, detecting can comprise detecting a change in dimension of a cell compartment of a disclosed component device.

In an aspect, changing can comprise changing a pharmacokinetic parameter selected from a group consisting of interactions between cells, liquid residence time, liquid to cell ratios, metabolism by cells, and shear stress in of a disclosed component device. In an aspect, changing can comprise changing a pharmacokinetic parameter selected from a group consisting of flow rate, chamber geometry, and number of cells in the component device.

In an aspect, a disclosed computerized method can comprise optimizing chamber geometry within the component device, wherein the optimizing includes selecting a quantity of chambers, choosing a chamber geometry that provides a proper tissue or organ size ratio, choosing an optimal fluid flow rate that provides a proper liquid residence time, and calculating a cell shear stress.

In an aspect, a disclosed computerized method can comprise regulating a temperature of the device or culture medium. In an aspect, a disclosed computerized method can comprise detecting fluorescent emissions from a cell compartment of a disclosed component device.

Disclosed herein is a computer-readable medium having computer-executable instructions stored thereon to perform a method, comprising, detecting data from one or more sensors in a component device, and analyzing data from one or more sensors to measure physiological events in one or more chambers of a component, and optionally, preparing a report of the data, regulating the device, or changing parameters of the device, cells, or a medium.

It is to be understood that this invention is not limited to specific nucleic acids, specific polypeptides, or to particular methods, as such can, of course, vary. It is also to be understood that the terminology used herein is for the purpose of describing particular embodiments only and is not intended to be limiting.

All patents, patent applications, and other referenced articles, journals or references referred to herein are each hereby expressly incorporated in its entirety.

As used in the specification and the appended claims, the singular forms “a,” “an” and “the” include plural referents unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance can or may not occur, and that the description includes instances where said event or circumstance occurs and instances where it does not.

As used herein, the term “determining” can refer to measuring or ascertaining an activity or an event or a quantity or an amount or a change in expression and/or in activity level or in prevalence and/or incidence. Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence. In an aspect, determining can refer to measuring the effect of one or more input variables on a culture system of cells. In an aspect, determining can refer to measure one or more output variables.

The term “contacting” as used herein refers to bringing a disclosed composition, a disclosed medium, a disclosed compound, or a disclosed complex together with an intended target (such as, e.g., a cell or population of cells, a receptor, an antigen, or other biological entity) in such a manner that the disclosed composition, compound, or complex can affect the activity of the intended target (e.g., receptor, transcription factor, cell, population of cells, etc.), either directly (i.e., by interacting with the target itself), or indirectly (i.e., by interacting with another molecule, co-factor, factor, or protein on which the activity of the target is dependent). For example, in an aspect, “contacting” can refer to contacting a disclosed culture analog system device with a medium, such as a serum-free medium.

As used herein, the term “analog” can refer to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. In an aspect, “analog” can refer to a system or an organ or a tissue that is sufficiently similar to a naturally occurring system or organ or tissue, such as a liver or the circulatory system.

As used throughout, by “subject” can be an individual. Preferably, the subject is a mammal such as a primate, and, more preferably, a human. Non-human primates include marmosets, monkeys, chimpanzees, gorillas, orangutans, and gibbons. The term “subject” includes domesticated animals, such as cats, dogs, etc., livestock (for example, cattle, horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). The subjects of the present invention can also include, but are not limited to fish (for example, zebrafish, goldfish, tilapia, salmon and trout), amphibians and reptiles.

Each of the following patent applications is herein incorporated by reference in its entirety: U.S. patent application Ser. No. 12/117,339 filed May 8, 2008 and titled “Culture of Electrically Functional Adult Spinal Cord Neurons and Associated Methods”; U.S. patent application Ser. No. 12/145,810 filed Jun. 25, 2008 and titled “Cell Culture Media and Process for Differentiation of Human Spinal Cord Stem Cells into Functional Motor Neuron Cells”; U.S. patent application Ser. No. 12/661,323 filed on Mar. 15, 2000 and titled “Bio-Microelectromechanical System Transducer and Associated Methods”; U.S. patent application Ser. No. 12/765,996 filed Apr. 23, 2010 and titled “Long Term In Vitro Culture of Tissue Engineered Functional Neuromuscular Junctions”; U.S. patent application Ser. No. 13/102,672 filed on May 6, 2011 and titled “Formation of Neuromuscular Junctions in a Defined System”; U.S. patent application Ser. No. 13/696,396 filed May 6, 2011 and titled “Formation of Neuromuscular Junctions”; U.S. patent application Ser. No. 12/765,399 filed on Apr. 22, 2010 and titled “Method for Culturing Skeletal Muscle for Tissue Engineering”; U.S. patent application Ser. No. 13/322,911 filed May 27, 2010 and titled “Method of Screening Drugs for Reversal of Amyloid Beta Neurotoxicity”; U.S. patent application Ser. No. 12/788,732 filed May 27, 2010 and titled “Method of Myelinating Isolated Motoneurons”; U.S. patent application Ser. No. 13/322,903 filed on May 28, 2010 and titled “In Vitro Production of Oligodendrocytes from Human Umbilical Cord Stem Cells”; U.S. patent application Ser. No. 12/938,701 filed Nov. 3, 2010 and titled “Patterned Cardiomyocyte Culture on Microelectrode Array”; U.S. patent application Ser. No. 13/576,442 filed Feb. 7, 2011 and titled “Model and Methods for Identifying Points of Action in Electrically Active Cells”; U.S. Provisional Patent Application No. 61/684,168 filed Aug. 17, 2012 and titled “Methods, Systems and Compositions for In Vitro Cellular Models of Mammalian Systems”; U.S. Provisional Patent Application Ser. No. 61/789,184 filed Mar. 15, 2013 and titled “Methods, Systems and Compositions for In Vitro Cellular Models of Mammalian Systems”; U.S. Provisional Patent Application No. 61/732,042 filed Nov. 30, 2012 and titled “Derivation of Sensory Neurons and Neural Crest Stem Cells from Human Neural Progenitor HNP1”; U.S. Provisional Patent Application No. 61/732,574 filed Dec. 3, 2012 and titled “Derivation of Sensory Neurons and Neural Crest Stem Cells from Human Neural Progenitor HNP1”; U.S. Provisional Patent Application Ser. No. 61/784,923 filed Mar. 14, 2013 and titled “Compositions and Methods for Generating Neural Crest Stem Cells”; U.S. Provisional Patent Application No. 61/758,628 filed Jan. 30, 2013 and titled “Compositions and Methods Comprising Cardiac Myocytes; and U.S. Provisional Patent Application Ser. No. 61/790,051 filed Mar. 15, 2013 and titled “Devices and Systems for Whole Heart Function”;

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by mole, temperature is in ° C. or is at ambient temperature, and pressure is at or near atmospheric.

### Example 1

The in vitro system disclosed herein can incorporate cells from at least two organs—for example, heart and liver. The cardiac cells were either harvested from neonatal rat cardiac tissue or obtained from commercial sources as differentiated human embryonic stem cells or adult induced pluripotent stem cells. A serum-free culture medium was formulated to promote both cell type growth and differentiation and was supplemented with specific growth factors (i.e., Epidermal growth factor (EGF)) or hormones (i.e., Hydrocortizone and L-Thyroxin) depending on the cell type.

As shown in FIG. 2A-FIG. 2B and FIG. 3A-FIG. 3B, the cardiomyocytes (either rat or stem cell derived) were plated in the inner chamber, while the HepG2 hepatocytes were plated in the outer chamber of the device. Since the cardiomyocytes were cultured on the top of micro-electrode arrays, their electrical activity was easily monitored and the cardiac toxicity induced by liver metabolites was assessed. Both cell types were incubated for 7-10 days in this device prior to signal recording, and representative recordings are shown in FIG. 2C and FIG. 3C.

TABLE 1
Composition of Medium 1

| Component | Amount | | Catalogue # | Source | References |
|---|---|---|---|---|---|
| Neurobasal | 500 | mL | 10888 | Gibco/Invitrogen | Brewer et al., 1993 |
| Antibiotic-Antimycotic | 5 | mL | 15240-062 | Gibco/Invitrogen | |
| G5 Supplement (100X) | 5 | mL | 17503-012 | Gibco/Invitrogen | Alterio et al., 1990; Clegg et al., 1987; Bottenstein 1981, |
| | | | | | 1988 (Science); Bottenstein et al., 1988; Morrow et al., |
| | | | | | 1990; Gonzalez et al., 1990; Moore et al., 1991; Anderson et |
| | | | | | al., 1991; Olwin et al., 1992 |
| VEGF_{165 r Human} | 10 | μg | P2654 | Gibco/Invitrogen | Arsic et al., 2004; Germani et al., 2003; Lee et al., 2003 |
| Acidic FGF | 12.5 | μg | 13241-013 | Gibco/Invitrogen | Alterio et al., 1990; Moore et al., 1991; Olwin et al., 1992; |
| | | | | | Motamed et al., 2003; Dusterhoft et al., 1999; Fu et al., |
| | | | | | 1995; Smith et al., 1994; Oliver et al., 1992; Dell'Era et al., |
| | | | | | 2003 |
| Heparin Sulphate | 50 | μg | D9809 | Sigma | Alterio et al., 1990; Moore et al., 1991; Olwin et al., 1992; |
| | | | | | Motamed et al., 2003; Dusterhoft et al., 1999; Fu et al., |
| | | | | | 1995; Smith et al., 1994; Oliver et al., 1992; Dell'Era et al., |
| | | | | | 2003 |
| LIF | 10 | μg | L5158 | Sigma | Husmann et al., 1996; Kurek et al., 1996; Megeney et al., |
| | | | | | 1996; Vakakis et al., 1995; Martinou et al., 1992; Sun et al., |
| | | | | | 2007; Malm et al., 2004; Zorzano et al., 2003; Sakuma et al., |
| | | | | | 2000 |
| Vitronectin (Rat Plasma) | 50 | μg | V0132 | Sigma | Biesecker et al., 1990; Gullberg et al., 1995 |
| CNTF | 20 | μg | CRC 401B | Cell Sciences | Wang et al., 2008; Chen et al., 2003, 2005; Cannon et al., |
| | | | | | 1998; Marques et al., 1997 |
| NT-3 | 10 | μg | CRN 500B | Cell Sciences | Oakley et al., 1997 |
| NT-4 | 10 | μg | CRN 501B | Cell Sciences | Carrasco et al., 2003; Simon et al., 2003 |
| GDNF | 10 | μg | CRG 400B | Cell Sciences | Choi-Lundberg et al., 1995; Lin et al., 1993; Yang et al., |
| | | | | | 2004; Golden et al., 1999; Henderson et al., 1994 |
| BDNF | 10 | μg | CRB 600B | Cell Sciences | Simon et al., 2003; Heinrich 2003; Mousavi et al., 2004 |
| CT-1 | 10 | μg | CRC 700B | Cell Sciences | Chen et al., 2004; Bordet et al., 2001; Dolcet et al., 2001; |
| | | | | | Lesbordes et al., 2002; Nishikawa et al., 2005; Mitsumoto et |
| | | | | | al, 2001; Oppenheim et al., 2001; Peroulakis et al., 2000; |
| | | | | | Sheng et al., 1996 |

TABLE 2
Composition of Medium 2

| Component | Amount | | Catalogue | Source | References |
|---|---|---|---|---|---|
| Neurobasal | 500 | mL | 10888 | Invitrogen/Gibco | Brewer et al., 1993 |
| Antibiotic-Antimycotic | 5 | mL | 15240-062 | Invitrogen/Gibco | |
| Cholesterol (250X) | 5 | mL | 12531 | Invitrogen/Gibco | Jaworska-Wilczynska et al., 2002 |
| TNF-alpha, human | 10 | μg | T6674 | Sigma-Aldrich | Caratsch et al., 1994; Al-Shanti et al., |
| | | | | | 2008; Fowler et al., 1993 |
| PDGF BB | 50 | μg | P4056 | Sigma-Aldrich | Husmann et al., 1996; Jin et al., 1991; |
| | | | | | Kudla et al, 1995; Quinn et al., 1990; |
| | | | | | Yablonka-Reuveni et al., 1995 |
| Vasoactive intestinal peptide (VIP) | 250 | μg | V6130 | Sigma-Aldrich | Gold et al., 1982 |
| Insulin-like growth factor 1 | 25 | μg | 12656 | Sigma-Aldrich | Malm et al., 2004; Zorzano et al., 2003; |
| | | | | | Al-Shanti et al., 2008 |
| NAP | 1 | mg | 61170 | AnaSpec. Inc. | Gozes et al., 2004; Aracil et al., 2004 |
| r-Apolipoprotein E2 | 50 | μg | P2002 | Panvera | Robertson et al., 2000 |
| Laminin, mouse purified | 2 | mg | 08-125 | Millipore | Langen et al., 2003; Foster et al., 1987; |
| | | | | | Hantai et al., 1991; Kuhl et al., 1986; Lyles |
| | | | | | et al., 1992; Song et al., 1992; Swasdison |
| | | | | | et al., 1992 |
| Beta amyloid (1-40) | 1 | mg | AG966 | Millipore | Wang et al., 2005; Yang et al., 2007; |
| | | | | | Akaaboune et al., 2000 |
| Human Tenascin-C protein | 100 | μg | CC065 | Millipore | Hall et al., 2000 |
| rr-Sonic hedgehog, Shh N- | 50 | μg | 1314-SH | R&D Systems | Brand-Saberi et al., 2005; Fan et al., 1994; |
| terminal | | | | | Munsterberg et al., 1995; |
| | | | | | Nelson et al., |
| | | | | | 1996; Cossu et al., 1996; Currie et al., |
| | | | | | 1996; Norris et al., 2000; Elia et al., 2007; |
| | | | | | Pagan et al., 1996; Holler et al., 2007; King |
| | | | | | et al., 2000; Lou et al., 2009 |
| rr (Agrin C terminal) | 50 | μg | 550-AG-100 | R&D Systems | Ku Mutyala et al.; Das et al., 2007 (Nature |
| | | | | | Protocols) |

TABLE 3
Serum-Free Culture Medium

| | Amount/ | | Catalog |
|---|---|---|---|
| Component | Concentration | Company | Number |
| Neurobasal E | 500 mL | Gibco | 10888 |
| B27 | 10 mL | Gibco | 17504-044 |
| G5 (100x) | 1 mL | Invitrogen | 17503-012 |
| aFGF | 10 μg | Invitrogen | 13241-013 |
| VEGF 165 | 10 μg | Invitrogen | P2654 |
| Human BDNF | 10 μg | Cell Sciences | CRB 600B |
| Human GDNF | 10 μg | Cell Sciences | CRG 400B |
| Rat CNTF | 25 μg | Cell Sciences | CRC 401B |
| Human CT-1 | 10 μg | Cell Sciences | CRC 700B |
| NT-3 | 10 μg | Cell Sciences | CRN 500B |
| NT-4 | 10 μg | Cell Sciences | CRN 501B |
| De-N-sulphated | 40 μg | Sigma | D9809 |
| N-acetylated heparin | | | |
| sulphate | | | |
| Vitronectin | 50 μg | Sigma | V0132 |
| Glutamax (100x) | 5 mL | Invitrogen | 35050-061 |
| Antibiotic-Antimycotic | 5 mL | Invitrogen | 15240-062 |
| 100x | | | |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Akaaboune M, et al. (2000) Developmental regulation of amyloid precursor protein at the neuromuscular junction in mouse skeletal muscle. Mol Cell Neurosci. 15(4):355-367.
Al-Shanti N, et al. (2008) Beneficial synergistic interactions of TNF•alpha and IL•6 in C2 skeletal myoblasts—potential cross-talk with IGF system. Growth Factors. 26(2):61-73.
Alterio J, et al. (1990) Acidic and basic fibroblast growth factor mRNAs are expressed by skeletal muscle satellite cells. Biochem Biophys Res Commun. 166(3):1205-1212.
Anderson J E, et al. (1991) Distinctive patterns of basic fibroblast growth factor (bFGF) distribution in degenerating and regenerating areas of dystrophic (mdx) striated muscles. Dev Biol. 147(1):96-109.
Aracil A, et al. (2004) Proceedings of Neuropeptides 2004, the XIV European Neuropeptides Club meeting. Neuropeptides. 38(6):369-371.
Arnold H H, et al. (1998) more complexity to the network of myogenic regulators. Curr Opin Genet Dev. 8(5):539-44.
Arsic N, et al. (2004) Vascular endothelial growth factor stimulates skeletal muscle regeneration in vivo. Mol. Ther. 10(5):844-854.
Bandi E, et al. (2008) Neural agrin controls maturation of the excitation-contraction coupling mechanism in human myotubes developing in vitro. Am J Physiol Cell Physiol. 294(1):C66-73.
Bers D M. Cardiac excitation-contraction coupling. Nature 2002; 415(6868): 198-205.
Biesecker G, et al. (1990) The complement SC5b•9 complex mediates cell adhesion through a vitronectin receptor. J. Immunol. 145(1):209-214.
Bordet T., et al. (2001) Protective effects of cardiotrophin-1 adenoviral gene transfer on neuromuscular degeneration in transgenic ALS mice. Hum Mol. Genet. 10(18):1925-1933.
Bottenstein J E, et al. (1981) Proliferation of glioma cells in serum-free defined medium. Cancer Treat Rep. 65 Suppl 2:67-70.
Bottenstein J E, et al. (1988) CNS neuronal cell line-derived factors regulate gliogenesis in neonatal rat brain cultures. J Neurosci Res. 20(3):291-303.
Bottenstein J E, et al. (1998) Advances in vertebrate cell culture methods. Science. 239(4841 Pt 2):G42, G48.
Brand-Saberi B, et al. (2005) Genetic and epigenetic control of skeletal muscle development. Ann Anat. 187(3):199-207.
Bren-Mattison Y, et al. (2002) Sonic hedgehog inhibits the terminal differentiation of limb myoblasts committed to the slow muscle lineage. Dev Biol. 242(2):130-148.
Brewer G J, et al. (1993) Optimized survival of hippocampal neurons in B27-supplemented Neurobasal, a new serum-free medium combination. J Neurosci Res. 35(5):567-576.
Brewer G J, et al. (2008) NbActiv4 medium improvement to Neurobasal/B27 increases neuron synapse densities and network spike rates on multielectrode arrays. J Neurosci Methods. 170(2):181-187.
Cannon J G, et al. (1998) Intrinsic and extrinsic factors in muscle aging. Ann NY Acad Sci. 854:72-77.
Caratsch C G, et al. (1994) Interferon-alpha, beta and tumor necrosis factor-alpha enhance the frequency of miniature end-plate potentials at rat neuromuscular junction. Neurosci Lett. 166(1):97-100.
Carlsson L. In vitro and in vivo models for testing arrhythmogenesis in drugs. Journal of Internal Medicine 2006; 259(1): 70-80.
Carrasco Dl, et al. (2003) Neurotrophin 4/5 is required for the normal development of the slow muscle fiber phenotype in the rat soleus. J Exp Bio. 206(Pt 13):2191-2200.
Chen J, et al. (2004) Role of exogenous and endogenous trophic factors in the regulation of extraocular muscle strength during development. Invest Ophthalmol V is Sci. 45(10):3538-3545.
Chen X, et al. (2005) Dedifferentiation of adult human myoblasts induced by ciliary neurotrophic factor in vitro. Mol Bioi Cell. 16(7):3140-3151.
Chen X P, et al. (2003) [Exogenous rhCNTF inhibits myoblast differentiation of skeletal muscle of adult human in vitro]. Sheng Li Xue Bao. 55(4):464-468.
Choi-Lundberg D L, et al. (1995) Ontogeny and distribution of glial cell line-derived neurotrophic factor (GDNF) mRNA in rat. Brain Res Dev Brain Res. 85(1):80-88.
Chow I, et al. (1985) Release of acetylcholine from embryonic neurons upon contact with muscle cell. J Neurosci. 5(4):1076-82.
Clegg C H, et al. (1987) Growth factor control of skeletal muscle differentiation: commitment to terminal differentiation occurs in G1 phase and is repressed by fibroblast growth factor. J Cell Biol. 105(2):949-956.
Colomar A, et al. (2004) Glial modulation of synaptic transmission at the neuromuscular junction. Glia. 47(3):284-9.
Cossu G, et al. (1996) How is myogenesis initiated in the embryo? Trends Genet. 12(6):218-223.
Currie P D, et al. (1996) Induction of a specific muscle cell type by a hedgehog-like protein in zebrafish. Nature. 382(6590):452-455.
Dakhel Y, et al. Erythomycinpotentiates pr interval prolonging effect of verapamil in the rat: A pharmacodynamic drug interaction. ToxicolApplPhamacol 2006; 214: 24-29.
Daniels M P, (1997) Intercellular communication that mediates formation of the neuromuscular junction. Mol Neurobiol. 14(3):143-170.
Daniels M P, et al. (2000) Rodent nerve-muscle cell culture system for studies of neuromuscular junction development: refinements and applications. Microsc Res Tech. 49(1):26-37.
Das M, et al. (2003) Electrophysiological and morphological characterization of rat embryonic motoneurons in a defined system. Biotechnol Prog. 19(6):1756-1761.
Das M, et al. (2004) Long-term culture of embryonic rat cardiomyocytes on an organosilane surface in a serum-free medium. Biomaterials. 25(25):5643-5647.
Das M, et al. (2005) Adult rat spinal cord culture on an organosilane surface in a novel serum-free medium. In Vitro Cell Dev Bioi Anim. 41(10):343-348.
Das M, et al. (2006) A defined system to allow skeletal muscle differentiation and subsequent integration with silicon microstructures. Biomaterials. 27(24):4374-4380.
Das M, et al. (2007) Auto-catalytic ceria nanoparticles offer neuroprotection to adult rat spinal cord neurons. Biomaterials. 28(10):1918-1925.
Das M, et al. (2007) Differentiation of skeletal muscle and integration of myotubes with silicon microstructures using serum-free medium and a synthetic silane substrate. Nat Protoc. 2(7): 1795-1801.
Das M, et al. (2007) Embryonic motoneuron-skeletal muscle co-culture in a defined system. Neuroscience. 146(2):481-488.
Das M, et al. (2008) Temporal neurotransmitter conditioning restores the functional activity of adult spinal cord neurons in long-term culture. Exp Neurol. 209(1): 171-180.
Das M, et al. (2009) Skeletal muscle tissue engineering: an improved model promoting long term survival of myotubes, structural development of e-c coupling apparatus and neonatal myosin heavy chain (MHC) expression. Biomaterials. 30:5392-402.
Das M, et al. A defined long-term in vitro tissue engineered model of neuromuscular junctions. Biomaterials 2010; 31: 4880-4888.
Dell'Era P, et al. (2003) Fibroblast growth factor receptor 1 is essential for in vitro cardiomyocyte development. Circ Res. 93(5):414-420.
Dhir V, et al. Patterning of diverse mammalian cell types in serum free medium with photoablation. Biotechnol Prog 2009; 25(2): 594-603.
Dolcet X, et al. (2001) Cytokines promote motoneuron survival through the Janus kinase-dependent activation of the phosphatidylinositol 3-kinase pathway. Mol Cell Neurosci. 18(6):619-631.
Dusterhoft S, et al. (1999) Evidence that acidic fibroblast growth factor promotes maturation of rat satellite-cell-derived myotubes in vitro. Differentiation. 65(3): 161-169.
Eisenberg T, et al. (2009) Induction of autophagy by spermidine promotes longevity. Nat Cell Biol. 11(11):1305-14.
Elia D, et al. (2007) Sonic hedgehog promotes proliferation and differentiation of adult muscle cells: Involvement of MAPK/ERK and P13K/Akt pathways. Biochim Biophys Acta. 1773(9):1438-1446.
English A W., et al. (2003) Cytokines, growth factors and sprouting at the neuromuscular junction. J Neurocytol. 32(5):943-60.
Fan C M, et al. (1994) Patterning of mammalian somites by surface ectoderm and notochord: evidence for sclerotome induction by a hedgehog homolog. Cell. 79(7):1175-1186.
Foster R F, et al. (1987) A laminin substrate promotes myogenesis in rat skeletal muscle cultures: analysis of replication and development using antidesmin and anti-BrdUrd monoclonal antibodies. Dev Bio. 122(1):11-20.
Fowler V M, et al. (1993) Tropomodulin is associated with the free (pointed) ends of the thin filaments in rat skeletal muscle. J Cell Biol. 120(2):411-420.
Fu X, et al. (1995) Acidic fibroblast growth factor reduces rat skeletal muscle damage caused by ischemia and reperfusion. Chin Med J (Engl). 108(3):209-214.
Germani A, et al. (2003) Vascular endothelial growth factor modulates skeletal myoblast function. Am J Pathol. 163(4):1417-1428.
Gold M R, et al. (1982) The effects of vasoactive intestinal peptide on neuromuscular transmission in the frog. J Physiol. 327:325-335.
Golden J P, et al. (1999) Expression of neurturin, GDNF, and GDNF family-receptor mRNA in the developing and mature mouse. Exp Neural. 158(2):504-528.
Gonzalez A M, et al. (1990) Distribution of basic fibroblast growth factor in the 18-day rat fetus: localization in the basement membranes of diverse tissues. J Cell Biol. 110(3):753-765.
Gozes I, et al. (2004) NAP mechanisms of neuroprotection. J Mol Neurosci. 24(1):67-72.
Gullberg D, et al. (1995) Analysis of fibronectin and vitronectin receptors on human fetal skeletal muscle cells upon differentiation. Exp Cell Res. 220(1):112-123.
Guo X F, et al. Characterization of a human fetal spinal cord stem cell line nsi-566rsc and its induction to functional motoneurons. Tissue Engin Regen Med 2010; 4: 181-193.
Guo X F, et al. NMJ formation between human stem cell derived motoneurons and rat skeletal muscle in a defined system. Tissue Engineering: Part C 2010; 16(6): 1347-1355.
Hall B K, et al. (2000) All for one and one for all: condensations and the initiation of skeletal development. Bioessays. 22(2): 138-147.
Hantai D, et al. (1991) Developmental appearance of thrombospondin in neonatal mouse skeletal muscle. Eur J Cell Biol. 55(2):286-294.
Heinrich G, et al. (2003) A novel BDNF gene promoter directs expression to skeletal muscle. BMC Neurosci. 4:11.
Henderson C E, et al. (1994) GDNF: a potent survival factor for motoneurons present in peripheral nerve and muscle. Science. 266(5187):1062-1064.
Holler F J, et al. (2007). Chapter 1. Principles of Instrumental Analysis (6th Edition ed.). Cengage Learning. p. 9, ISBN 9780495012016.
Hughes B. 2007 FDA drug approvals: A year of flux specialty products dominate innovative drug approvals—a trend that looks set to continue. Nature Reviews Drug Discovery 2008; 7: 107-109.
Husmann I, et al. (1996) Growth factors in skeletal muscle regeneration. Cytokine Growth Factor Rev. 7(3):249-258.
Jaworska-Wilczynska M, et al. (2002) Three lipoprotein receptors and cholesterol in inclusion-body myositis muscle. Neurology. 58(3):438-445.
Jin P, et al. (1991) Recombinant platelet-derived growth factor-88 stimulates growth and inhibits differentiation of rat L6 myoblasts. J Biol Chem. 266(2):1245-1249.
Jung D R, et al. Cell-based sensor microelectrode array characterized by imaging x-ray photoelectron spectroscopy, scanning electron microscopy, impedance measurements, and extracellular recordings. J Vac Sci Technol A 1998; 16(3): 1183-1188.
Kim C, et al. Non-cardiomyocytes influence the electrophysiological maturation of human embryonic stem cell-derived cardiomyocytes during differentiation. Stem cells and development 2010; 19(6): 783-795.
King T, et al. (2000). Piezoactuators for ‘real-world’ applications—Can they deliver sufficient displacement? Power Engineering J, 20(3): 105-110.
Kita-Matsuo H, et al. Lentiviral vectors and protocols for creation of stable hesc lines for fluorescent tracking and drug resistance selection of cardiomyocytes. PLoS ONE 2009; 4(4): e5046.
Koleva M, et al. (2005) Pleiotropic effects of sonic hedgehog on muscle satellite cells. Cell Mol Life Sci. 62(16): 1863-1870.
Kudla A J, et al. (1995) A requirement for fibroblast growth factor in regulation of skeletal muscle growth and differentiation cannot be replaced by activation of platelet-derived growth factor signaling pathways. Mol Cell Biol. 15(6):3238-3246.
Kuhl U, et al. (1986) Role of laminin and fibronectin in selecting myogenic versus fibrogenic cells from skeletal muscle cells in vitro. Dev Biol. 117(2):628-635.
Kurek J B, et al. (1996) Leukemia inhibitory factor and interleukin-6 are produced by diseased and regenerating skeletal muscle. Muscle Nerve. 19(10):1291-1301.
Langen R C, et al. (2003) Enhanced myogenic differentiation by extracellular matrix is regulated at the early stages of myogenesis. In Vitro Cell Dev Bioi Anim. 39(3-4): 163-169.
Lawrence C L, et al. Nonclinical proarrhythmia models: Predicting torsades de pointes. Journal of Pharmacological and Toxicological Methods 2005; 52(1): 46-59.
Lee E W., et al. (2003) Neuropeptide Y induces ischemic angiogenesis and restores function of ischemic skeletal muscles. J Clin Invest. 111(12): 1853-1862.
Lesbordes J C, et al. (2002) In vivo electrotransfer of the cardiotrophin-1 gene into skeletal muscle slows down progression of motor neuron degeneration in pmn mice. Hum Mol Genet. 11(14):1615-1625.
Lescaudron L, et al. (1999) Blood borne macrophages are essential for the triggering of muscle regeneration following muscle transplant. Neuromuscul Disord. 9(2):72-80.
Li M X, et al. (2001) Opposing actions of protein kinase A and C mediate Hebbian synaptic plasticity. Nat Neurosci. 4(9):871-872.
Lin L F, et al. (1993) GDNF: a glial cell line-derived neurotrophic factor for midbrain dopaminergic neurons. Science. 260(5111):1130-1132.
Liu W P, et al. Enantioselectivity in environmental safety of current chiral insecticides. Proc Natl Acad Sci USA 2005; 102(3): 701-706.
Lou X J. (2009) Polarization fatigue in ferroelectric thin films and related materials. 105, 024101-1.
Lou X J. (2009) Statistical switching kinetics of ferroelectrics. J Phys Condens Matter. 21(1):012207.
Lund A E, et al. Dose-dependent interaction of the pyrethroid isomers with sodium-channels of squid axon-membranes. Neurotoxicology 1982; 3(1): 11-24.
Lyles J M, et al. (1992) Matrigel enhances myotube development in a serum-free defined medium. Int J Dev Neurosci. 10(1):59-73.
Maduell F. Hemodiafiltration. Hemodial Int 2005; 9(1): 47-55.
Mahler G J, et al. Characterization of a gastrointestinal tract microscale cell culture analog used to predict drug toxicity. Biotechnol Bioeng 2009; 104(1): 193-205.
Mahler G J, et al. Characterization of caco-2 and ht29-mtx co-cultures in an in vitro digestion/cell culture model used to predict iron bioavailability. J Nutr Biochem 2009; 20(7): 494-502.
Maim C, et al. (2004) Leukocytes, cytokines, growth factors and hormones in human skeletal muscle and blood after uphill or downhill running J Physiol. 556(Pt 3):983-1000.
Marona H R N, et al. Determination of sparfloxcin and its degradation products by hplc-pda. J Antimicrob Chemother 1999; 44: 301-302.
Marques M J, et al. (1997) Ciliary neurotrophic factor stimulates in vivo myotube formation in mice. Neurosci Lett. 234(1):43-46.
Martinou J C, et al. (1992) Cholinergic differentiation factor (CDF/LIF) promotes survival of isolated rat embryonic motoneurons in vitro. Neuron. 8(4):737-744.
Mayes L, et al. (2007) Pbx homeodomain proteins direct Myod activity to promote fast-muscle differentiation. Development. 134(18):3371-3382.
McAuliffe G J, et al. Development of a gastrointestinal tract microscale cell culture analog to predict drug transport. Mol Cell Bioengr 2008; 5(2): 119-132.
Megeney L A, et al. (1996) bFGF and LIF signaling activates STAT3 in proliferating myoblasts. Dev Genet. 19(2):139-145.
Meyer T, et al. Qt-screen: High-throughput cardiac safety pharmacology by extracellular electrophysiology on primary cardiac myocytes. Assay and Drug Development Technologies 2004; 2(5): 507-514.
Miller S C, et al. (1998) Tumor necrosis factor inhibits human myogenesis in vitro. Mol Cell Biol. 8(6):2295-2301.
Mitsumoto H, et al. (2001) Effects of cardiotrophin•1 (CT-1) in a mouse motor neuron disease. Muscle Nerve. 24(6):769-777.
Mohan D K, et al. Toxin detection based on action potential shape analysis using a realistic mathematical model of differentiated ng108-15 cells. Biosens Bioelectron 2006; 21: 1804-1811.
Molnar P, et al. Photolithographic patterning of c2c12 myotubes using vitronectin as growth substrate in serum-free medium. BiotechnolProg 2007; 23(1): 265-268.
Molnar P, et al. Synaptic connectivity in engineered neuronal networks, in Patch-clamp methods and protocols, Molnar P and Hickman J J, Editors. 2007, Humana Press: New York.
Moore J W, et al. (1991) The mRNAs encoding acidic FGF, basic FGF and FGF receptor are coordinately downregulated during myogenic differentiation. Development. 111(3):741-748.
Morrow N G, et al. (1990) Increased expression of fibroblast growth factors in a rabbit skeletal muscle model of exercise conditioning. J Clin Invest. 85(6):1816-1820.
Motamed K, et al. (2003) Fibroblast growth factor receptor-1 mediates the inhibition of endothelial cell proliferation and the promotion of skeletal myoblast differentiation by SPARC: a role for protein kinase A. J Cell Biochem. 90(2):408-423.
Mousavi K, et al. (2004) BDNF rescues myosin heavy chain 118 muscle fibers after neonatal nerve injury. Am J Physiol Cell Physicl. 287(1):C22-29.
Mufti N A, et al. Different in vitro systems affect cyp1a1 activity in response to 2,3,7,8-tetrachlorodibenzo-p-dioxin. Toxicol in vitro 1998; 12: 259-272.
Munsterberg A E, et al. (1995) Combinatorial signaling by Sonic hedgehog and Wnt family members induces myogenic bHLH gene expression in the somite. Genes Dev. 9(23):2911-2922.
Mutayla M S K, et al. Mechanical and electronic approaches to improve the sensitivity of microcantilever sensors. Acta Mech Sin, 25:1-12.
Nakamura S, et al. Analysis of cardiac toxicity caused by cyclophosphamide in the H9c2 cell line and isolated and perfused rat hearts. Gan To Kagaku Ryoho 2010; 37(4):677-680.
Nakamura Y, et al. The in vitro metabolism of a pyrethroid insecticide, permethrin, and its hydrolysis products in rats. Toxicol Appl Pharmacol 2007; 235: 176-184.
Natarajan A, et al. Multielectrode recordings of cardiac action potentials as a high throughput method to evaluate pesticide toxicity. Toxicol In Vitro 2006; 20(3): 375-381.
Natarajan A, et al. Patterned cardiomyocytes on microelectrode arrays as a functional, high information content drug screening platform. Biomaterials 2011; in press.
Nelson C E, et al. (1996) Analysis of Hox gene expression in the chick limb bud. Development. 122(5):1449-1466.
Nelson PG (1975) Nerve and muscle cells in culture. Physiol Rev. 55(1):1-61.
Nishikawa J, et al. (2005) Increase of Cardiotrophin-1 immunoreactivity in regenerating and overloaded but not denervated muscles of rats. Neuropathology. 25(1): 54-65.
Norris W, et al. (2000) Slow muscle induction by Hedgehog signalling in vitro. J Cell Sci. 113 (Pt 15):2695-2703.
Oakley R A, et al. (1997) Neurotrophin-3 promotes the differentiation of muscle spindle afferents in the absence of peripheral targets. J Neurosci. 17(11):4262-4274.
Oh T-i, et al. Real-time fluorescence detection of multiple microscale cell culture analog devices in situ. Cytometry Part A 2007; 71A: 857-865.
Oliver L, et al. (1992) Acidic fibroblast growth factor (aFGF) in developing normal and dystrophic (mdx) mouse muscles. Distribution in degenerating and regenerating mdx myofibres. Growth Factors. 7(2):97-106.
Olson E N., et al. (1992) Interplay between proliferation and differentiation within the myogenic lineage. Dev Biol. 154(2):261-72.
Olwin B B, et al. (1992) Repression of myogenic differentiation by aFGF, bFGF, and K•FGF is dependent on cellular heparan sulfate. J Cell Biol. 118(3):631-639.
Oppenheim R W, et al. (2001) Cardiotrophin-1, a muscle-derived cytokine, is required for the survival of subpopulations of developing motoneurons. J Neurosci. 21(4):1283-1291.
Pagan S M, et al. (1996) Surgical removal of limb bud Sonic hedgehog results in posterior skeletal defects. Dev Biol. 180(1):35-40.
Peroulakis M E, et al. (2000) Forger NG: Ciliary neurotrophic factor increases muscle fiber number in the developing levator ani muscle of female rats. Neurosci Lett. 296(2-3):73-76.
Pointer C, et al. Ht29-mtx and caco-21tc7 monolayers as predictive models for human intestional absorption: Role of mucus layer. J PharmSci 2001; 90: 1608-1619.
Quinn L S, et al. (1990) Paracrine control of myoblast proliferation and differentiation by fibroblasts. Dev Biol. 140(1):8-19.
Ravenscroft M S, et al. (1998) Developmental Neurobiology Implications from Fabrication and Analysis of Hippocampal Neuronal Networks on Patterned Silane-Modified Surfaces. J Am Chem Soc. 120(47):12169-12177.
Robertson T A, et al. (2000) Comparison of astrocytic and myocytic metabolic dysregulation in apolipoprotein E deficient and human apolipoprotein E transgenic mice. Neuroscience. 98(2):353-359.
Rumsey J W, et al. Node of ranvier formation on motoneurons in vitro. Biomaterials 2009; 30: 3567-3572.
Sakuma K, et al. (2000) Differential adaptation of growth and differentiation factor 8/myostatin, fibroblast growth factor 6 and leukemia inhibitory factor in overloaded, regenerating and denervated rat muscles. Biochim Biophys Acta. 1497(1):77-88.
Sandi E, et al. (2008) Neural agrin controls maturation of the excitation-contraction coupling mechanism in human myotubes developing in vitro. Am J Physiol, Cell Physiol. 294(1):C66-73.
Sanes J R, et al. (1997) Genetic analysis of postsynaptic differentiation at the vertebrate neuromuscular junction. Curr Opin Neurobiol. 7(1):93-100.
Schaffner A E, et al. Investigation of the factors necessary for growth of hippocampal neurons in a defined system. J Neurosci Methods 1995; 62(1-2): 111-9.
Scollon E J, et al. In vitro metabolism of pyrethroid pesticides by rat and human hepatic microsomes and cytochrome p450 isoforms. Drug Metabolism and Disposition 2009; 37(1): 221-228.
Selivanova O M, et al. Compact globular structure of thermusthermophilus ribosomal protein s1 in solution. J BiolChem 2003; 278(38): 36311-36314.
Sheng Z, et al. (1996) Cardiotrophin-1 displays early expression in the murine heart tube and promotes cardiac myocyte survival. Development. 122(2):419-428.
Simon M, et al. (2003) Effect of NT-4 and BDNF delivery to damaged sciatic nerves on phenotypic recovery of fast and slow muscles fibres. Eur J Neurosci. 18(9):2460-2466.
Sin A, et al. The design and fabrication of three-chamber microscale cell culture analog devices with integrated dissolved oxygen sensors. Biotechnol Prog 2004; 20: 338-345.
Smith J, et al. (1994) The effects of fibroblast growth factors in long-term primary culture of dystrophic (mdx) mouse muscle myoblasts. Exp Cell Res. 210(1):86-93.
Song W K, et al. (1992) H36-alpha 7 is a novel integrin alpha chain that is developmentally regulated during skeletal myogenesis. J Cell Biol. 117(3):643-657.
Stenger D A, et al. (1992) Coplanar molecular assemblies of aminoalkylsilane and perfluorinated alkylsilane—characterization and geometric definition of mammalian-cell adhesion and growth. J Am Chem Soc. 114(22):8435-42.
Sun L, et al. (2007) JAK1-STAT1.-STAT3, a key pathway promoting proliferation and preventing premature differentiation of myoblasts. J Cell Biol. 179(1): 129-138.
Sung J H, et al. A micro cell culture analog with 3-d hydrogel culture of multiple cell lines to assess metabolism-dependent cytotoxicity of anti-cancer drugs. Lab Chip 2009; 9(10): 1385-1394.
Sung J H, et al. A microfludic device for a pharmacokinetic-pharmacodynamic (pk-pd) model on a chip. Lab Chip 2010; 10: 446-455.
Sung J H, et al. Fluorescence optical detection in situ for real time monitoring of enzymatic activity of liver cells in multiple microfludic devices. Biotechnol Bioeng 2009; 104: 516-525.
Sung J H, et al. Prevention of air bubble formation in a microfluidic perfusion cell culture system using a microscale bubble trap. Biomed Microdevices 2009; 11: 731-738.
Suter W. Predictive value of in vitro safety studies. Current Opinion in Chemical Biology 2006; 10(4): 362-366.
Sutton N M, et al. Clinical effects and outcome of feline permethrin spot-on poisonings reported to the veterinary poisons information service (vpis), london. J Feline Med & Surgery 2007; 9: 335-339.
Swasdison S, et al. (1992) Formation of highly organized skeletal muscle fibers in vitro. Comparison with muscle development in vivo. J Cell Sci. 102 (Pt 3):643-652.
Swynghedauw B. Molecular mechanisms of myocardial remodeling. Physiol Rev 1999; 79(1): 215-262.
Takagishi Y, et al. Species-specific difference in distribution of voltage-gated 1-type ca2+ channels of cardiac myocytes. Am J Physiol Cell Physiol 2000; 279(6): C1963-1969.
Tatosian D A, et al. A novel system for evaluation of drug mixtures for potential efficacy in treating multidrug resistant cancers. Biotechnol Bioeng 2009; 103(1): 187-198.
Torgan C E, et al. (2001) Regulation of myosin heavy chain expression during rat skeletal muscle development in vitro. Mol Bioi Cell 12(5): 1499-1508.
Torgan C E, et al. (2006) Calcineurin localization in skeletal muscle offers insights into potential new targets. J Histochem Cytochem. 54(1): 119-128.
Vakakis N, et al. (1995) In vitro myoblast to myotube transformations in the presence of leukemia inhibitory factor. Neurochem Int. 27(4-5):329-335.
Varghese K, et al. A new target for amyloid beta toxicity validated by standard and high-throughput electrophysiology. PLoS One 2010; 5(1): e8643.
Varghese K, et al. Regeneration and characterization of adult mouse hippocampal neurons in a defined in vitro system. J Neurosci Methods 2009; 177: 51-59.
Viravaidya K, et al. Incorporation of 3t3-11 cells to mimic bioaccumulation in a microscale cell culture analog device for toxicity studies. Biotechnol Prog 2004; 20: 590-597.
Vogel Z, et al. (1976) Ultrastructure of acetylcholine receptor clusters on cultured muscle fibers. J Cell Biol. 69(2):501-507.
Wang P, et al. (2005) Defective neuromuscular synapses in mice lacking amyloid precursor protein (APP) and APP-Like protein 2. J Neurosci. 25(5): 1219-1225.
Wang X, et al. (2008) Effects of interleukin-6, leukemia inhibitory factor, and ciliary neurotrophic factor on the proliferation and differentiation of adult human myoblasts. Cell Mol Neurobiol. 28(1): 113-124.
Wilson K, et al. Direct patterning of coplanar polyethylene glycol alkylsilane monolayers by deep-ultraviolet photolithography as a general method for high fidelity, long-term cell patterning and culture. JVST B 2011; in press.
Wilson K, et al. Integration of functional myotubes with a bio-mems device for non-invasive interrogation. Lab Chip 2007; 7: 920-922.
Wilson K, et al. Measurement of contractile stress generated by cultured muscle on silicon cantilevers. PLoS One 2010; 5(6): e11042.
Witzemann V., (2006) Development of the neuromuscular junction. Cell Tissue Res. 326(2):263-71.
Xu H, et al. Development of a stable dual cell-line GFP expression system to study estrogenic endocrine disruptors. Biotechnol Bioeng 2008; 101(6): 1276-1287.
Yablonka-Reuveni Z, et al. (1995) Development and postnatal regulation of adult myoblasts. Microsc Res Tech. 30(5):366-380.
Yang L, et al. (2007) Increased asynchronous release and aberrant calcium channel activation in amyloid precursor protein deficient neuromuscular synapses. Neuroscience. 149(4):768-778.
Yang L X, et al. (2004) Glia cell line-derived neurotrophic factor regulates the distribution of acetylcholine receptors in mouse primary skeletal muscle cells. Neuroscience. 128(3):497-509.
Zimmermann W H, et al. Tissue engineering of a differentiated cardiac muscle construct. Circulation Research 2002; 90(2): 223-230.
Zorzano A, et al. (2003) Intracellular signals involved in the effects of insulin-like growth factors and neuregulins on myofibre formation. Cell Signal. 15(2):141-149.

What is claimed is:

1. An array of components, comprising:
one or more culture analog system devices comprising one or more chambers and one or more sensing elements, wherein a chamber simulates at least one interorgan interaction, physiological condition, physical condition, chemical condition, or electrical condition of one or more whole organs, tissues, or organisms.

2. The array of claim 1, wherein the one or more chambers comprises cells.

3. The array of claim 1, further comprising connection elements, filters, sensors, alarms, and computer control elements.

4. The array of claim 1, wherein the at least one interorgan interaction, physiological condition, physical condition, chemical condition, or electrical condition is monitored in real time.

5. The array of claim 2, wherein the cells are derived from a human, an animal, a plant or an insect, or combinations and mixtures thereof.

6. The array of claim 5, wherein the cells are derived from a human.

7. The array of claim 1, wherein the one or more culture analog system devices comprises a chip comprising cells on a microelectrode array comprising surface embedded microelectrodes.

8. The array of claim 2, wherein the one or more culture analog system devices comprises at least a first chamber comprising a first type of cell under conditions where the first type of cell provides at least one parameter value comparable to a value obtained for the same type of cell in vivo, wherein the first chamber is concentrically contained within a second chamber containing a second type of cell under conditions where the second type of cell provides at least one parameter value comparable to a value obtained for the same type of cell in vivo.

9. The array of claim 8, wherein the second chamber is concentrically contained within a third chamber containing a third type of cell under conditions where the third type of cell provides at least one parameter value comparable to a value obtained for the same type of cell in vivo.

10. The array of claim 9, wherein the third chamber is concentrically contained within a fourth chamber containing a fourth type of cell under conditions where the fourth type of cell provides at least one parameter value comparable to a value obtained for the same type of cell in vivo.

11. The array of claim 10, wherein the fourth chamber is concentrically contained within a fifth chamber containing a fifth type of cell under conditions where the fifth type of cell provides at least one parameter value comparable to a value obtained for the same type of cell in vivo.

12. The array of claim 2, wherein the one or more culture analog system devices comprises a first chamber comprising a first cell type maintained under conditions providing at least one parameter value comparable to values obtained for the cells in vivo; a second chamber of the same or different geometry than the first chamber comprising a second cell type maintained under conditions providing at least one parameter value comparable to values obtained for the cells in vivo; wherein the first and second chambers are interconnected by a fluidic connection about the chamber wall of the first chamber.

13. The array of claim 12, further comprising one or more additional chambers containing the same or different types of cells as in the first or second chambers, under conditions where the same or different types of cell provide at least one parameter value comparable to a value obtained for the same type of cell in vivo, wherein the one or more additional chambers are interconnected by a fluidic connection about the chamber walls of the interior chambers.

14. The array of claim 1, further comprising at least one culture medium.

15. The array of claim 2, wherein the at least one interorgan interaction, physiological condition, physical condition, chemical condition, or electrical condition is measured by determining one or more reactions by cells to an active agent, one or more reactions by cells to an input parameter, interaction between cells, liquid residence time, liquid to cell ratio, metabolism by cells, or shear stress.

16. A method for determining the effect of an input variable on a culture system of cells, comprising:
contacting at least one cell of a component or an array of components with an input variable,
wherein the component or the array of components comprise one or more culture analog system devices comprising one or more chambers and one or more sensing elements, wherein a chamber simulates at least one interorgan interaction, physiological condition, physical condition, chemical condition, or electrical condition of one or more whole organs, tissues, or organisms; and
monitoring at least one output parameter.

17. The method of claim 16, wherein monitoring at least one output parameter comprises obtaining information from a sensor in a chamber, chip or region, or from one or more devices in the array.

18. The method of claim 16, wherein the input variable is an organic compound, an inorganic compound, a complex sample, a pharmaceutical compound, an environmental sample, a nutritional sample, a consumer product, a virus, a liposome, a nanoparticle, a biodegradable polymer, a radiolabeled particle or toxin, a biomolecule, a toxin-conjugated particle, or a biomolecule.

19. The method of claim 16, wherein the method comprises high throughput studies or chronic toxicity studies.

20. The method of claim 16, wherein the method is executed for 72 hours, 84 hours, 96 hours, 108 hours, 120 hours, 132 hours, 144 hours, 156 hours, 168 hours, 180 hours, for days, or for weeks.
